(19) United States
(12) Patent Application Publication
(10) Pub. No.: US 2013/0281999 A1
(43) Pub. Date: Oct. 24, 2013

(54) METHOD OF PERFORMING MICROBEAM RADIOSURGERY

(71) Applicants: John R. ADLER, STANFORD, CA (US); Michael Dean WRIGHT, PALO ALTO, CA (US)

(72) Inventors: John R. ADLER, STANFORD, CA (US); Michael Dean WRIGHT, PALO ALTO, CA (US)

(73) Assignee: VARIAN MEDICAL SYSTEMS, INC., PALO ALTO, CA (US)

(21) Appl. No.: 13/453,338

(22) Filed: Apr. 23, 2012

(51) Int. Cl. A61B 18/18

(52) U.S. Cl. 606/33

(57) ABSTRACT

A method of performing microbeam radiosurgery on a patient whereby target tissue within a patient is irradiated with high energy electromagnetic radiation from an inverse Compton scattering radiation source via microbeam envelopes.

### BACKGROUND

1. Field of the Invention

The present invention relates to methods for performing radiosurgery on a patient, and in particular, to methods for performing radiosurgery using microbeam radiation.

2. Description of the Related Art

For over a century, high energy radiation (e.g., X- and y-radiation) has been used to destroy cancerous tumors located deep within the bodies of patients. This form of cancer therapy, known as radiotherapy, is one of the three major methods for treating cancer, surgery and chemotherapy being the remaining two. Radiotherapy is widely used. Indeed, nearly 60% of all cancer patients receive radiotherapy as an element of their overall treatment protocols.

Recently, radiation has also been used to treat non-cancerous tissues which are otherwise diseased or compromised. A particularly exciting emerging medical protocol utilizes radiation to either destroy or modulate the function of brain tissue associated with psychiatric or neurological disorders. Such treatments hold the promise of curing problems such as depression, chronic pain, and obesity.

The use of radiation to treat all forms of disease and biological dysfunction is known as radiosurgery.

Conventional radiosurgery employs three methods to generate high energy radiation. In a first method, the physical phenomenon of radioactivity is used. In a second method, the physical phenomenon of bremsstrahlung (i.e., “braking radiation,” arising from decelerating charged particles) is used. In a third method, the physical phenomenon of oscillating charged particles is used.

Conventional radiosurgery systems also generate three types of radiation spatial patterns with which to expose tissue. In a first case, the spatial pattern is uniform, and is described as a broad or non-segmented beam. In a second case, the spatial pattern is comprised of a two dimensional array of substantially mutually parallel circular or rectangular beams, and is described as a grid or segmented beam. In a third case, the spatial pattern is comprised of a linear array of substantially mutually parallel rectangular beams, and is described as a segmented beam. If the diameter of the circular beams, or the width of the rectangular beams, is less than 1 mm, such beams are described as microbeams.

Referring to FIG. 1, in the physical process of radioactivity for the nuclide ⁶⁰Co, a neutron of the ⁶⁰Co nucleus emits a β⁻ particle 10 (a.k.a., an electron), leaving behind an also radioactive ⁶⁰Ni nuclide. The activate ⁶⁰Ni nucleus in turn emits two high energy γ-ray photons 12 and 14 at 1.17 and 1.33 MeV, respectively, yielding a stable ⁶⁰Ni nuclide.

Referring to FIG. 2, a conventional radiosurgery system uses the radioactive nuclide ⁶⁰Co. The ⁶⁰Co material 20 is placed in the hollow portion of an otherwise solid Pb sphere 22. A patient is irradiated with photons 12, 14 when a slide mechanism 24 brings the ⁶⁰Co material 20 into position over a channel 26 within the Pb sphere 22 which is aligned with the patient (not shown). A collimator 28 between the ⁶⁰Co material 20 and the patient shapes the radiation field to provide either a broad or segmented beam.

Referring to FIG. 3, in the physical process of bremsstrahlung, a high energy electron 30 inelastically scatters off the nucleus 32 of a target atom, such as W. In the collision with the target nucleus 32, the electron 30 decelerates and loses energy. Some of the energy lost by the electron 30 emerges from the collision as a high energy X-ray photon 34.

Referring to FIG. 4, a conventional radiosurgery system which employs bremsstrahlung uses a linear accelerator 40 to provide a beam of high energy electrons 42 which is directed at a W target 44. High energy X-ray photons 34 emerge from the W target 44. A collimator 28 between the W target 44 and the patient (not shown) shapes the radiation field to provide either a broad or segmented beam.

Referring to FIG. 5, the generation of radiation via the oscillation of a charged particle is shown. An electron 50 is made to oscillate between two points A and B in space. As a result of this oscillation, a photon 52 emerges.

Referring to FIGS. 6A-6B, in a conventional radiosurgery system using the oscillation of charged particles to produce high energy radiation, a linear accelerator 40 (FIG. 6A) provides a beam of high energy electrons 42 which is injected into a synchrotron 60. The output of the synchrotron 60 is, in turn, injected into a storage ring 62. Located along a portion of the storage ring 62 circumference is a device known as a wiggler 64. The wiggler 64 (FIG. 6B) includes a series of magnets 66 providing an oscillating magnetic field pattern. As the electrons 50 move through the wiggler 64, the electrons 50 oscillate in a plane perpendicular to the plane of the oscillating magnetic field. The oscillating electrons 50, in turn, produce high energy radiation 52 (FIG. 6A) which is directed at a patient (not shown). A collimator 28 yields either a broad or segmented beam.

Referring to FIGS. 7A-7C, the three types of radiation spatial patterns typically used by conventional radiosurgery systems are depicted: a broad, non-segmented beam (FIG. 7A), a grid segmented pattern with a two dimensional array of substantially mutually parallel circular beams (FIG. 7B), and a segmented pattern with a linear array of substantially mutually parallel rectangular beams (FIG. 7C). If the individual beam dimensions 70, 71, 72 are less than 1 mm, the associated beam is considered a microbeam.

A major difficulty presented by these conventional radiosurgery systems is that the radiation which destroys diseased tissue also destroys normal healthy tissue. For most conventional radiosurgery systems, this problem is dealt with by exposing the diseased tissue from several angles, thereby maximizing the dose to the diseased tissue while minimizing the dose to neighboring normal tissue. Even so, the maximum dose which can be deposited in the diseased tissue, which determines the effectiveness of the radiation in destroying the diseased tissue, is limited by the susceptibility of the neighboring normal tissue to damage.

As indicated in Slatkin et al., U.S. Pat. No. 5,339,347 (the disclosure of which is incorporated herein by reference), experiments show that microbeam radiation patterns essentially resolve the problem of damage to normal tissue. Although the normal cells in the direct path of the microbeams are destroyed, the region of destroyed cells is so narrow that the healthy cells on either side are capable of healing the damaged region of tissue. Furthermore, as shown in Dilmanian et al., U.S. Pat. No. 7,194,063 (the disclosure of which is incorporated herein by reference), there exist microbeam targeting strategies which assure the destruction of diseased tissue while sparing the functionality of neighboring normal tissue.

One problem that can disannul the effectiveness of microbeam radiosurgery, however, is tissue movement during irradiation. Such movement may arise from patient breathing, or the pulsing of blood through the tissue. Movement of the tissue effectively broadens the regions irradiated by the microbeams. As the irradiated regions become wide, the healing capability of surrounding tissue is compromised. To avoid this problem, the microbeam radiation is preferably delivered extremely quickly so that the range of tissue motion during the irradiation is sufficiently small. Thus, the radiation source providing the microbeams preferably has a high dose rate.

Of the conventional radiosurgery systems described herein (FIGS. 2, 4 and 6A-6B), only the synchrotron source utilizing oscillating charged particles (FIGS. 6A-6B) has the ability to provide a sufficiently high dose rate to assure the effectiveness of microbeam radiosurgery. At the current state of the art, a synchrotron source has a maximum dose rate of nearly 2×10⁴ Gy/s, while a linear accelerator utilizing bremsstrahlung (FIG. 4) has a maximum dose rate of 4×10⁻¹ Gy/s, and a ⁶⁰Co source utilizing radioactivity (FIG. 2) has a maximum dose rate of 7×10⁻² Gy/s. These dose rates must be compared against the dose rate required to successfully treat the most challenging problem presented to microbeam radiosurgery, that of a moving lung tumor. A minimum dose rate of 7×10³ Gy/s is required to ablate a lung tumor using microbeam radiation.

Unfortunately, a synchrotron is a very large and expensive device. The synchrotron source which has been used for most microbeam radiosurgery experiments to date is the European Synchrotron Radiation Facility located in Grenoble, France. The storage ring associated with this synchrotron is 300 m in diameter, and the facility cost approximately $900M to construct. These characteristics of a synchrotron source prohibit widespread use of microbeam radiosurgery.

### SUMMARY

In accordance with the presently claimed invention, microbeam radiosurgery is performed by irradiating target tissue within a patient with high energy electromagnetic radiation from an inverse Compton scattering radiation source via microbeam envelopes.

In accordance with one embodiment of the presently claimed invention, a method of performing microbeam radiosurgery on a patient includes irradiating a target tissue, within a patient, with high energy electromagnetic radiation from an inverse Compton scattering radiation source via a plurality of microbeam envelopes which are mutually spatially distinct.

### BRIEF DESCRIPTION OF THE DRAWINGS

FIG. 1 depicts an energy level diagram for radioactivity for the nuclide ⁶⁰Co.

FIG. 2 depicts a conventional radiosurgery system using the radioactive nuclide ⁶⁰Co.

FIG. 3 depicts the physical process of bremsstrahlung.

FIG. 4 depicts a conventional radiosurgery system using bremsstrahlung.

FIG. 5 depicts the generation of radiation via the oscillation of a charged particle.

FIGS. 6A-6B depict a conventional radiosurgery system using the oscillation of charged particles to produce high energy radiation.

FIGS. 7A-7C depict three types of radiation spatial patterns typical of conventional radiosurgery systems.

FIG. 8 depicts inverse Compton scattering.

FIG. 9 depicts one example of a radiation source using inverse Compton scattering.

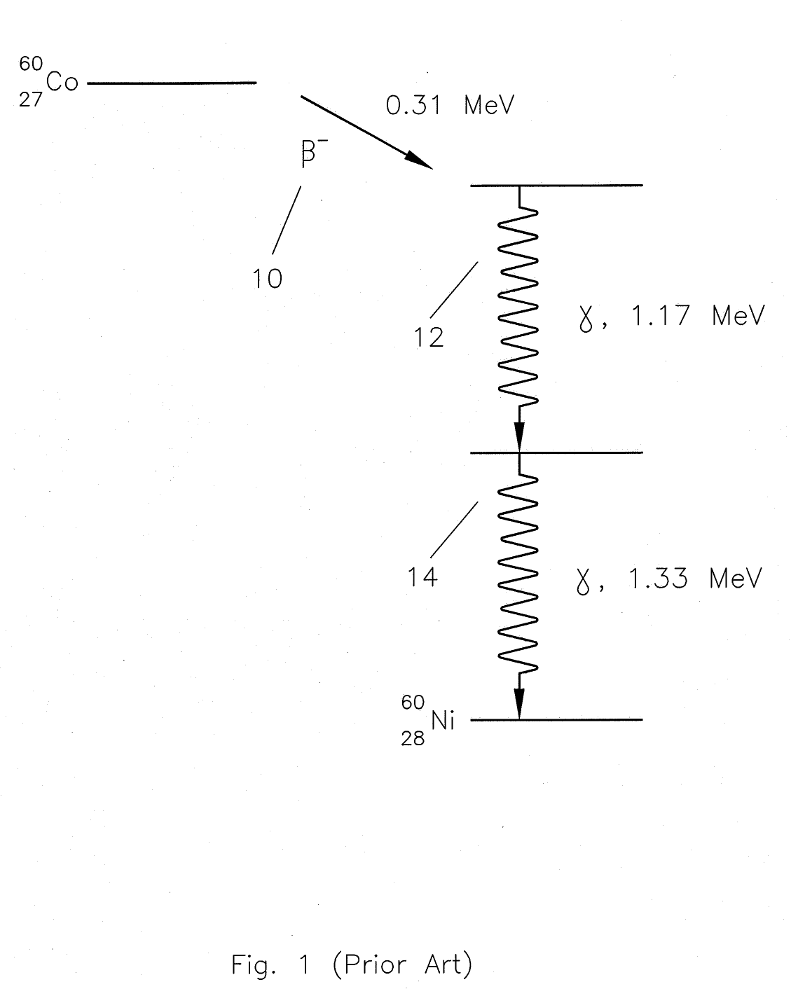

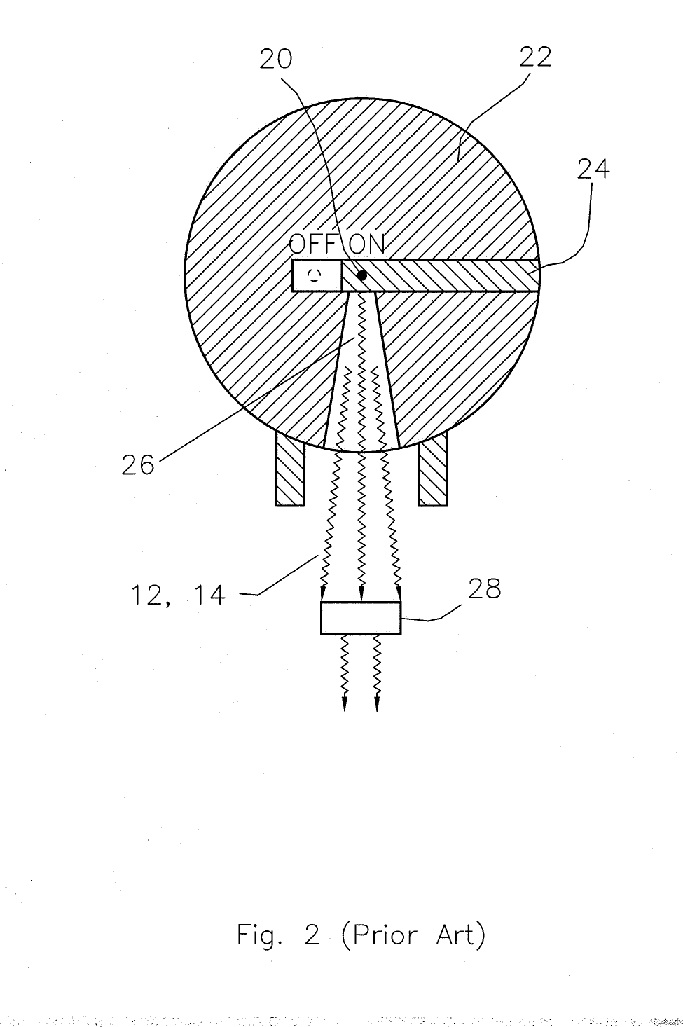

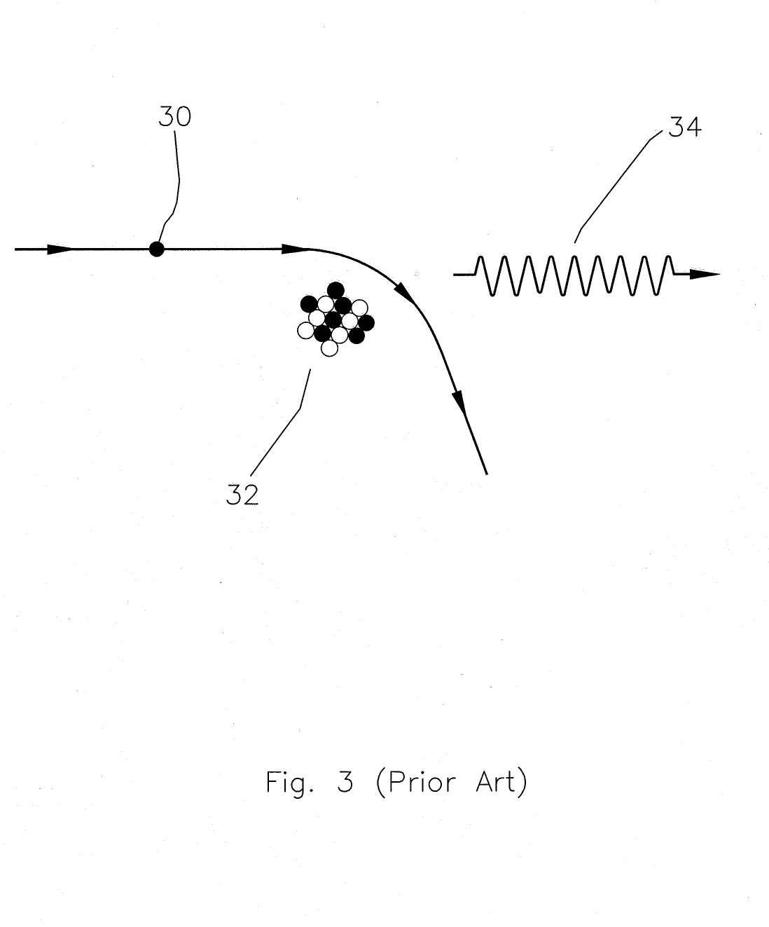

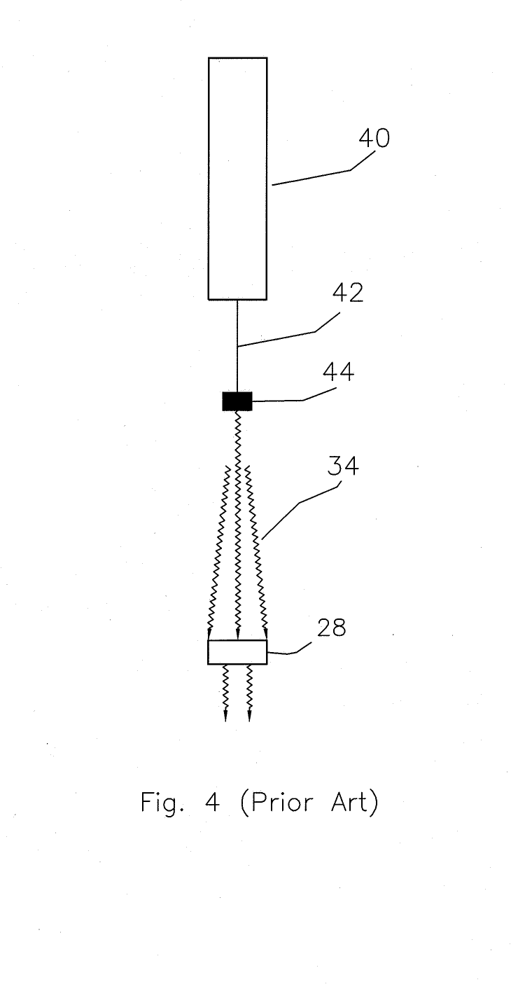

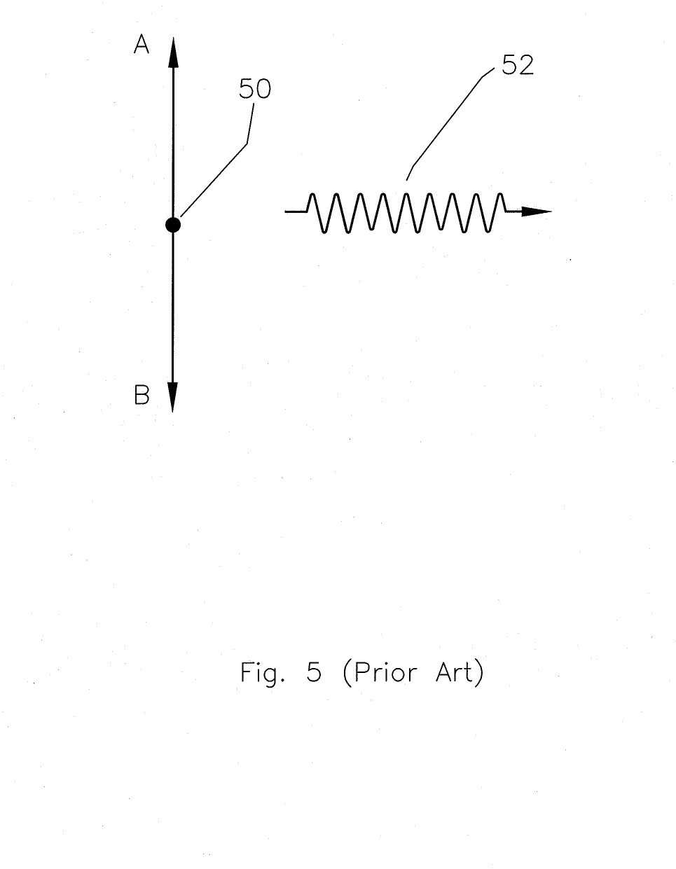

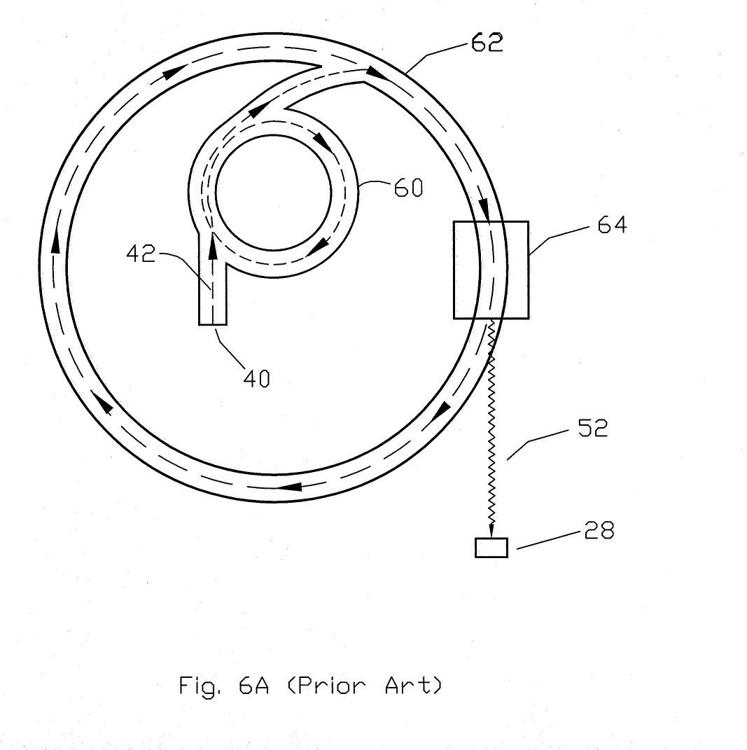

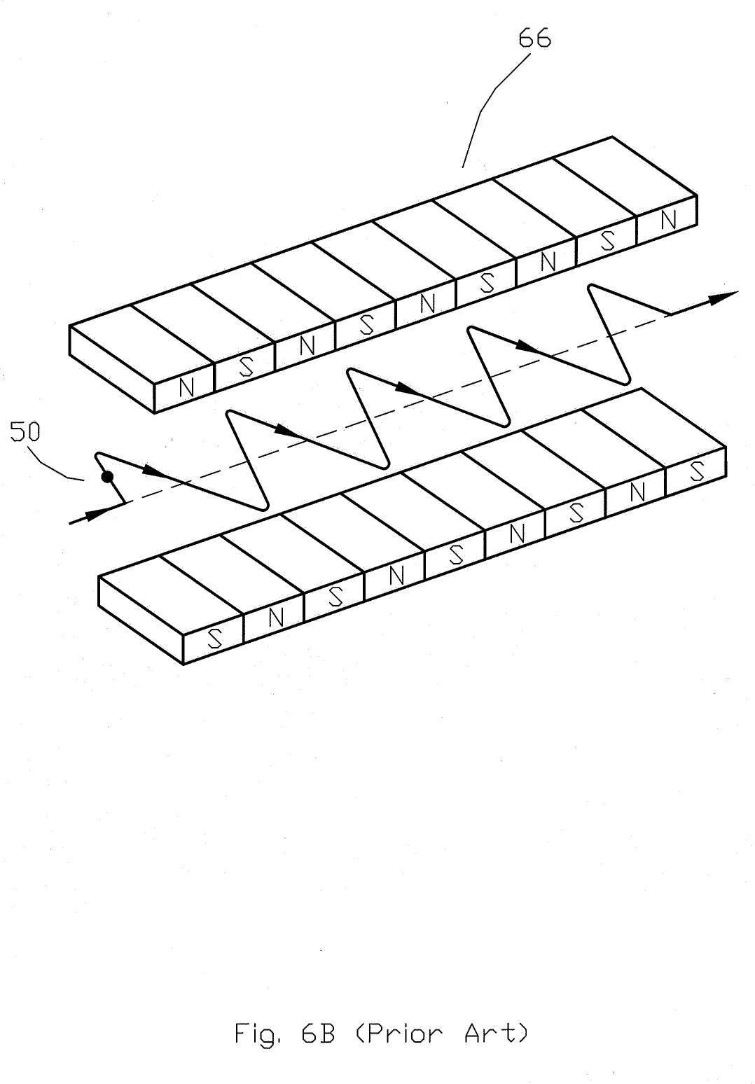

### DETAILED DESCRIPTION

The following detailed description is of example embodiments of the presently claimed invention with references to the accompanying drawings. Such description is intended to be illustrative and not limiting with respect to the scope of the present invention. Such embodiments are described in sufficient detail to enable one of ordinary skill in the art to practice the subject invention, and it will be understood that other embodiments may be practiced with some variations without departing from the spirit or scope of the subject invention.

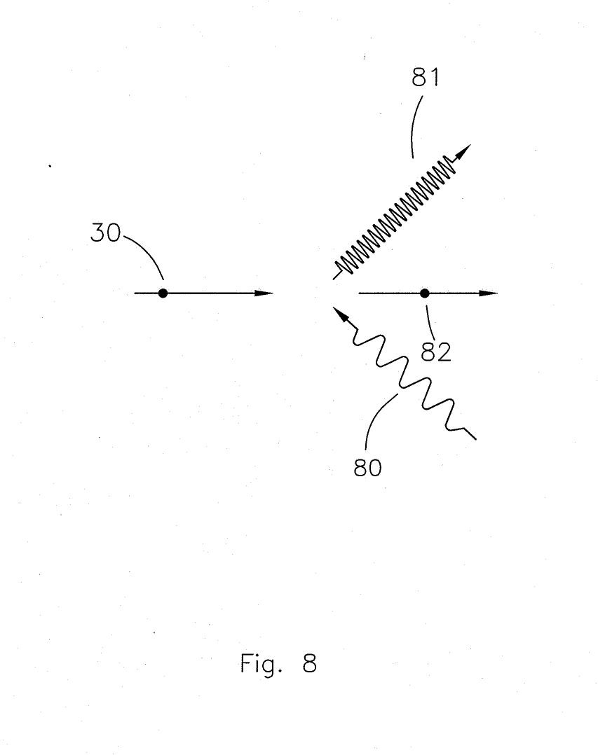

Referring to FIG. 8, radiosurgery using microbeam radiation in accordance with a preferred embodiment uses the physical process of inverse Compton scattering in which a high energy electron 30 collides with a low energy photon 80. Emerging from the collision is a high energy photon 81 and a reduced energy electron 82.

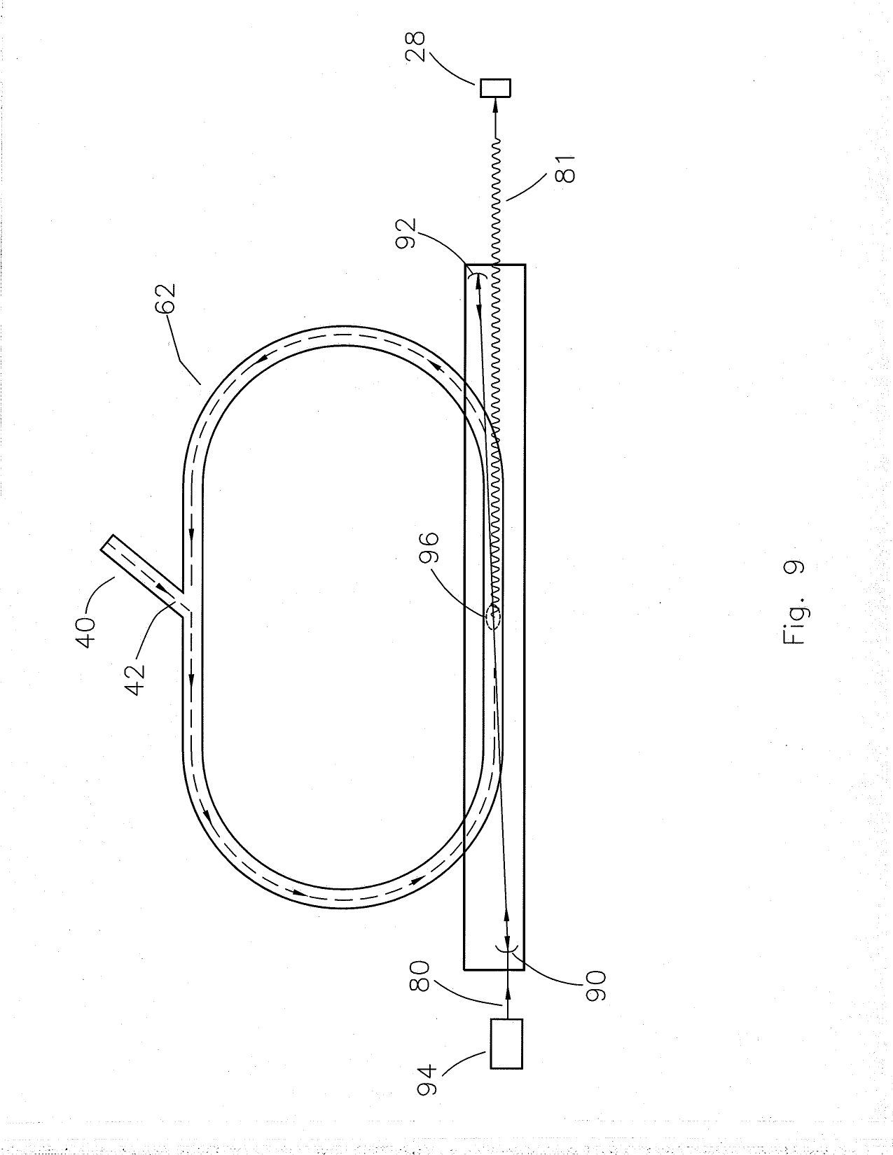

Referring to FIG. 9, in accordance with an exemplary embodiment, a radiation source utilizing inverse Compton scattering useful for microbeam radiosurgery includes a linear accelerator 40 which injects pulses of high energy electrons 42 into a small storage ring 62. The electron beam path along a portion of the storage ring 62 is substantially collinear with an optical cavity established by two mirrors 90, 92. Light from a pulsed, mode-locked laser 94 is injected into the optical cavity. The repetition rate of the laser 94 is set such that the pulses of laser light arrive at an interaction region 96 at the same time as the pulses of high energy electrons 42. As the high energy electrons collide with the low energy laser photons 80, high energy photons 81 are generated.

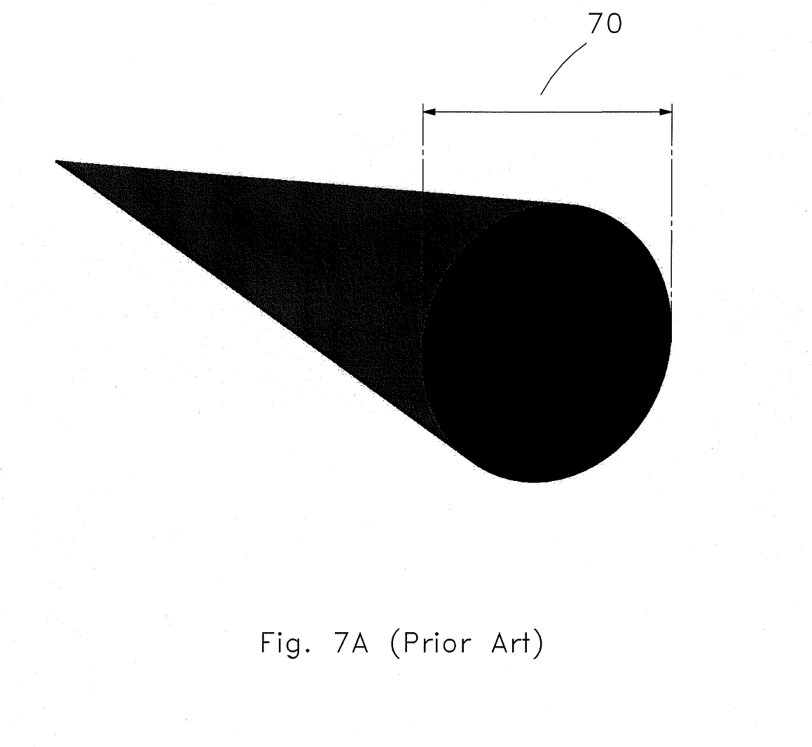

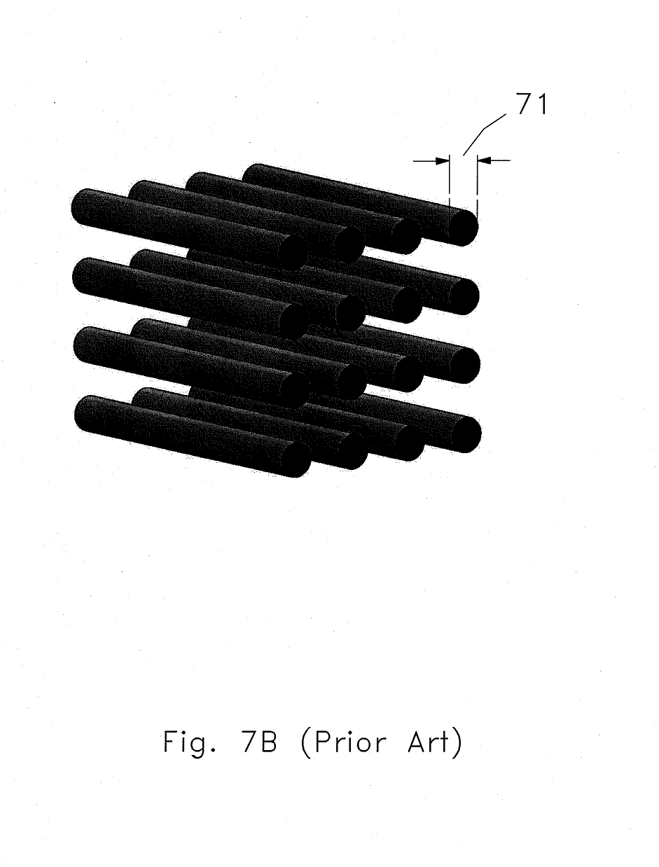

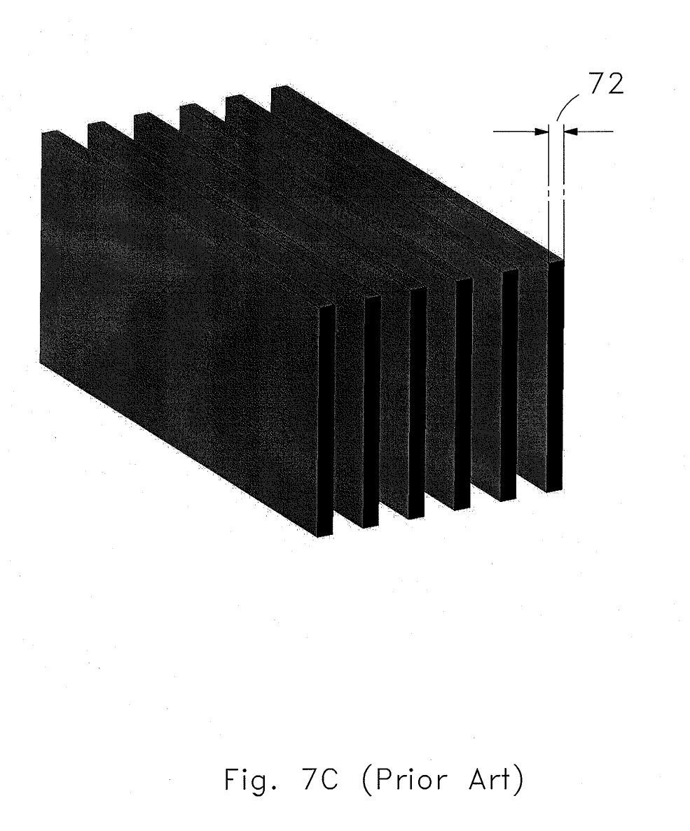

The high energy photons 81 can be arranged into the desired pattern of one or more microbeams (e.g., as depicted in FIGS. 7A-7C) in accordance with various techniques. For example, they can be passed through a collimator 28 which segments the radiation into the desired one or more simultaneous microbeams. For another example, the track of the electron beam 42 circulating in the storage ring 62 (FIG. 9) and/or the track of the low energy photon beam 80 circulating in the optical cavity defined by the mirrors 90, 92 can be manipulated to produce a beam of high energy photons 81 which scans through the desired regions of space as a function of time.

An inverse Compton scattering source of radiation such as described above should achieve a dose delivery rate of 1×10⁴ Gy/s. The diameter of the storage ring associated with such a source is expected to be less than 10 m, and the cost of such a source is expected to be less than $15 M.

What is claimed is:

1. A method of performing microbeam radiosurgery on a patient, comprising:
irradiating a target tissue, within a patient, with high energy electromagnetic radiation from an inverse Compton scattering radiation source via a plurality of microbeam envelopes which are mutually spatially distinct.

2. The method of claim 1, wherein said plurality of microbeam envelopes comprises a plurality of simultaneous microbeam envelopes.

3. The method of claim 1, wherein said plurality of microbeam envelopes comprises at least first and second portions provided sequentially in time.

4. The method of claim 1, wherein other tissue between adjacent ones of said plurality of microbeam envelopes support recovery of non-target tissue.

5. The method of claim 1, wherein said plurality of microbeam envelopes comprises a plurality of substantially mutually parallel microbeam envelopes.

6. The method of claim 1, further comprising generating said high energy electromagnetic radiation with an inverse Compton scattering radiation source.

7. The method of claim 6, further comprising collimating said high energy electromagnetic radiation to provide said plurality of microbeam envelopes.

8. The method of claim 6, wherein said generating said high energy electromagnetic radiation with an inverse Compton scattering radiation source comprises generating said high energy electromagnetic radiation with a storage ring.

9. The method of claim 6, wherein said generating said high energy electromagnetic radiation with an inverse Compton scattering radiation source comprises generating said high energy electromagnetic radiation with a laser light source.

10. The method of claim 6, wherein said generating said high energy electromagnetic radiation with an inverse Compton scattering radiation source comprises generating said high energy electromagnetic radiation with a linear accelerator.

11. The method of claim 1, wherein each of said plurality of microbeam envelopes has a lateral dimension of less than one millimeter.
